(19) United States
(12) Patent Application Publication
(10) Pub. No.: US 2024/0176982 A1
(43) Pub. Date: May 30, 2024

(54) TRAINING GRAPH NEURAL NETWORKS USING A DE-NOISING OBJECTIVE

(71) Applicant: DeepMind Technologies Limited, London (GB)

(72) Inventors: Jonathan William Godwin, London (GB); Peter William Battaglia, London (GB); Kevin Michael Schaarschmidt, Cambridge (GB); Alvaro Sanchez, London (GB)

(21) Appl. No.: 18/283,131

(22) PCT Filed: May 30, 2022
(86) PCT No.: PCT/EP2022/064565

§ 371 Date: Sep. 20, 2023

Related U.S. Application Data
(60) Provisional application No. 63/194,851, filed on May 28, 2021.

(51) Int. Cl. G06N 3/04; G06N 3/084

(52) U.S. Cl. CPC G06N 3/04; G06N 3/084

(57) ABSTRACT

Methods, systems, and apparatus, including computer programs encoded on a computer storage medium, for training a neural network that includes one or more graph neural network layers. In one aspect, a method comprises: generating data defining a graph, comprising: generating a respective final feature representation for each node, wherein, for each of one or more of the nodes, the respective final feature representation is a modified feature representation that is generated from a respective feature representation for the node using respective noise; processing the data defining the graph using one or more of the graph neural network layers of the neural network to generate a respective updated node embedding of each node; and processing, for each of one or more of the nodes having modified feature representations, the updated node embedding of the node to generate a respective de-noising prediction for the node.

### CROSS-REFERENCE TO RELATED APPLICATIONS

This application claims the benefit of the filing date of U.S. Provisional Patent Application Ser. No. 63/194,851 for “TRAINING GRAPH NEURAL NETWORKS USING A DE-NOISING OBJECTIVE,” which was filed on May 28, 2021, and which is incorporated herein by reference in its entirety.

### BACKGROUND

This specification relates to processing data using machine learning models.

Machine learning models receive an input and generate an output, e.g., a predicted output, based on the received input. Some machine learning models are parametric models and generate the output based on the received input and on values of the parameters of the model.

Some machine learning models are deep models that employ multiple layers of models to generate an output for a received input. For example, a deep neural network is a deep machine learning model that includes an output layer and one or more hidden layers that each apply a non-linear transformation to a received input to generate an output.

### SUMMARY

This specification generally describes a training system implemented as computer programs on one or more computers in one or more locations that trains a neural network that includes one or more graph neural network layers.

As used throughout this specification, a “graph” refers to a data structure that includes at least: (i) a set of nodes, and (ii) a set of edges. Each edge in the graph can connect a respective pair of nodes in the graph. The graph can be a “directed” graph, i.e., such that each edge that connects a pair of nodes is defined as pointing from the first node to the second node or vice versa, or an “undirected” graph, i.e., such that the edges (or pairs of oppositely directed edges) are not associated with directions.

Generally, data defining a graph can include data defining the nodes and the edges of the graph, and can be represented in any appropriate numerical format. For example, a graph can be defined by data including a listing of tuples {(i, j)} where each tuple (i, j) represents an edge in the graph connecting the node i and node j. Moreover, each edge in the graph can be associated with a set of one or more edge features, and each node in the graph can be associated with a set of one or more node features.

In one aspect there is described a method for training a neural network that includes one or more graph neural network layers. The method comprises generating data defining a graph that comprises: (i) a set of nodes, (ii) a node embedding for each node, and (iii) a set of edges that each connect a respective pair of nodes. In implementations this comprises obtaining a respective initial feature representation for each node and generating a respective final feature representation for each node, where, for each of one or more of the nodes, the respective final feature representation is a modified feature representation that is generated from the respective feature representation for the node using respective noise, and generating the data defining the graph using the respective final feature representations of the nodes. In implementations the node embedding for each node is generated from the respective final feature representation of the node.

The method processes the data defining the graph using one or more of the graph neural network layers of the neural network to generate a respective updated node embedding of each node. The method processes, for each of one or more of the nodes having modified feature representations, the updated node embedding of the node to generate a respective de-noising prediction for the node that characterizes a de-noised feature representation for the node that does not include the noise used to generate the modified feature representation of the node. The method determines an update to current values of neural network parameters of the neural network to optimize an objective function that measures errors in the de-noising predictions for the nodes, in particular to optimize the respective de-noising predictions for the nodes.

In some implementations, for each of one or more of the nodes having modified feature representations, the respective de-noising prediction for the node predicts the noise used to generate the modified feature representation of the node.

In some implementations, for each of one or more of the nodes having modified feature representations, the respective de-noising prediction for the node predicts the respective initial feature representation of the node.

In some implementations, for each of one or more of the nodes having modified feature representations, the respective de-noising prediction for the node characterizes a target feature representation of the node.

In some implementations, for each of one or more of the nodes having modified feature representations, the respective de-noising prediction for the node predicts an incremental feature representation for the node that, if added to the modified feature representation for the node, results in the target feature representation of the node.

In some implementations, the method further comprises processing the updated node embeddings of the nodes to generate a task prediction, wherein the objective function also measures an error in the task prediction.

In some implementations, both: (i) the updated node embeddings of the nodes, and (ii) original node embeddings of the nodes prior to being updated using the graph neural network layers, are processed to generate the task prediction.

In some implementations, the graph represents a molecule and the task prediction is a prediction of an equilibrium energy of the molecule.

In some implementations, the objective function measures, for each of a plurality of graph neural network layers of the neural network, respective errors in de-noising predictions for the nodes that are based on updated node embeddings generated by the graph neural network layer.

In some implementations, for each of one or more of the nodes having modified feature representations, processing the updated node embedding of the node to generate the respective de-noising prediction for the node comprises: processing the updated node embedding of the node using one or more neural network layers to generate the respective de-noising prediction for the node.

In some implementations, determining the update to the current values of the neural network parameters of the neural network to optimize the objective function comprises: backpropagating gradients of the objective function through neural network parameters of the graph neural network layers.

In some implementations, for each of one or more of the nodes, the respective final feature representation for the node is generated by adding the respective noise to the respective feature representation for the node.

In some implementations, generating the data defining the graph using the respective final feature representations of the nodes comprises: determining, for each pair of nodes comprising a first node and a second node, a respective distance between the final feature representation for the first node and the final feature representation for the second node; and determining that each pair of nodes corresponding to a distance that is less than a predefined threshold are connected by an edge in the graph.

In some implementations, the graph further comprises a respective edge embedding for each edge.

In some implementations, generating the data defining the graph comprises: generating an edge embedding for each edge in the graph based at least in part on a difference between the respective final feature representations of the nodes connected by the edge.

In some implementations, the graph represents a molecule, each node in the graph represents a respective atom in the molecule, and generating the data defining the graph comprises: generating a node embedding for each node based on a type of atom represented by the node.

In some implementations, the neural network includes at least 10 graph neural network layers.

In some implementations, each graph neural network layer of the graph neural network is configured to: receive a current graph; and update the current graph in accordance with current neural network parameter values of the graph neural network layer, comprising: updating a current node embedding of each of one or more nodes in the graph based on: (i) the current node embedding of the node, and (ii) a respective current node embedding of each of one or more neighbors of the node in the graph.

In some implementations, the current graph comprises an edge embedding for each edge, and updating the current node embedding of each of one or more nodes in the graph further comprises: updating the node embedding of the node based at least in part on a respective edge embedding of each of one or more edges connected to the node.

In some implementations, the graph represents a molecule, each node in the graph represents a respective atom in the molecule, the initial feature representation for each node represents an initial spatial position of a corresponding atom in the molecule, and for each of one or more of the nodes having modified feature representations, and the target feature representation for the node represents a final spatial position of the corresponding atom after atomic relaxation.

According to another aspect there are provided one or more non-transitory computer storage media storing instructions that when executed by one or more computers cause the one or more computers to perform operations of the methods described herein.

According to another aspect there is provided a system comprising: one or more computers; and one or more storage devices communicatively coupled to the one or more computers, wherein the one or more storage devices store instructions that, when executed by the one or more computers, cause the one or more computers to perform operations of the methods described herein.

As used throughout this specification, an “embedding” refers to an ordered collection of numerical values, e.g., a vector, matrix, or other tensor of numerical values.

The subject matter described in this specification can be implemented in particular embodiments so as to realize one or more of the following advantages.

The system described in this specification can train a graph neural network (i.e., a neural network that includes one or more graph neural network layers) to generate de-noising predictions for the nodes in the graph. In particular, prior to training the graph neural network on a graph, the system can modify feature representations of the nodes in the graph using noise, e.g., by adding noise to the feature representations of the nodes. The de-noising predictions can, e.g., predict the values of the noise that modified the feature representations of the nodes, or predict a reconstruction of the original feature representations of the nodes in the graph (i.e., before the feature representations were modified using the noise).

Training the graph neural network to generate de-noising predictions can regularize the training of the graph neural network, and in particular, can enable effective training of graph neural networks with large numbers of graph neural network layers, e.g., more than 100 graph neural network layers. In contrast, many conventional systems are limited to training graph neural networks having far fewer graph neural network layers (e.g., fewer than 10 layers) before the performance of the graph neural network saturates or even decreases with the addition of more graph neural network layers. Deeper graph neural networks (i.e., having more graph neural network layers), when trained by the system described in this specification, can achieve higher prediction accuracy for more complex prediction tasks than would be achievable using shallower graph neural networks (i.e., having fewer graph neural network layers).

Generating de-noising predictions requires each node embedding to encode unique information in order to de-noise the feature representation of the node, which can mitigate the effects of “over-smoothing,” e.g., where the node embeddings become nearly identical after being processed through a number of graph neural network layers. Moreover, training the graph neural network to generate de-noising predictions can reduce the likelihood of “over-fitting,” e.g., because the noise added to the feature representations of the nodes in the graph prevents the graph neural network from memorizing the original node feature representations. Training the graph neural network to generate de-noising predictions also encourages the graph neural network to implicitly learn the distribution of “real” graphs, i.e., with unmodified node feature representations, and the graph neural network can leverage this implicit knowledge to achieve higher accuracy on “task” predictions. Because the described techniques work differently to other techniques that involve dropping node or edge features they can be combined with these other techniques. The system described in this specification thus enables more efficient use of computational resources (e.g., memory and computing power) by enabling effective training of deeper graph neural networks achieving higher accuracy while mitigating the effects of over-smoothing and over-fitting.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a block diagram of an example training system for training a graph neural network.

FIG. 2 illustrates an example of operations that can be performed by the training system.

FIG. 3 is a flow diagram of an example process for using a training system to train a graph neural network.

FIG. 4, FIG. 5, and FIG. 6 illustrate example experimental results.

Like reference numbers and designations in the various drawings indicate like elements.

### DETAILED DESCRIPTION

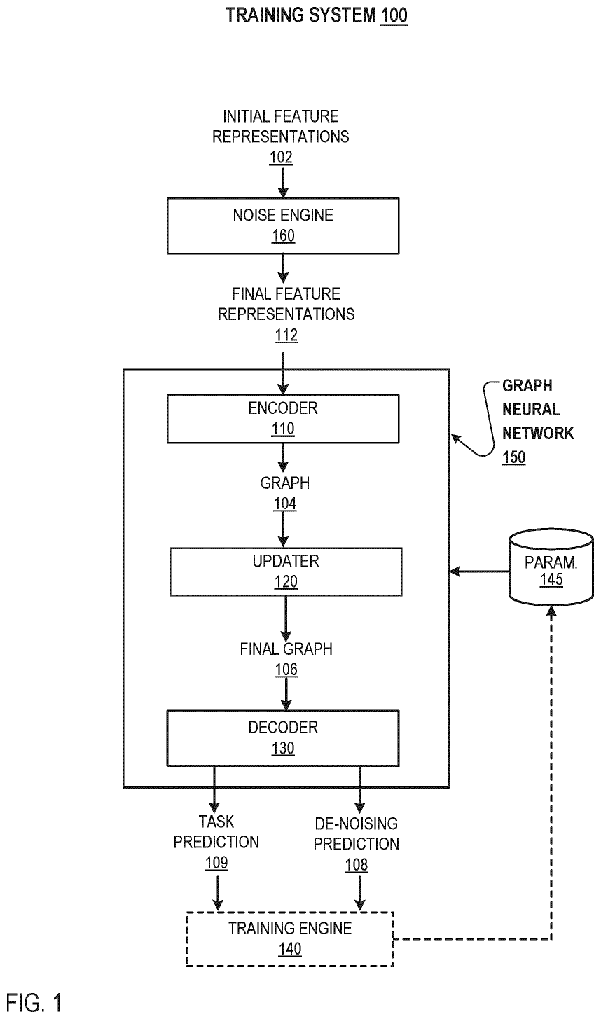

FIG. 1 is a block diagram of an example training system 100 for training a graph neural network 150, e.g., a neural network that includes one or more graph neural network layers. The system 100 is an example of a system implemented as computer programs on one or more computers in one or more locations in which the systems, components, and techniques described below are implemented.

The system 100 can train the graph neural network 150 by using the graph neural network 150 to generate de-noising predictions 108 for nodes in a graph 104. Generally, a “graph” refers to a data structure that includes at least: (i) a set of nodes, and (ii) a set of edges. For example, the graph can be represented as, e.g., G=(V, E), where V is the set of nodes and E is the set of edges. Each edge in the graph can connect a pair of nodes in the graph. In some implementations, the graph G can additionally be represented using graph-level properties g, e.g., G=(V, E, g), where the graph-level properties g can include any appropriate aspect of the overall system represented by the graph 104. As a particular example, the graph 104 can represent a physical system, e.g., a molecule, each node in the graph 104 can represent, e.g., an atom in the molecule, and graph level properties can include, e.g., an energy of the molecule. Generally, the graph 104 can represent any appropriate type of system, e.g., a collection of particles, a point cloud, or a social network. Examples of systems that can be represented by the graph 104 are described in more detail below.

The training system 100 can include a noise engine 160 that can be configured to process an initial feature representation 102 for each node in the graph 104 and generate respective final feature representation 112 for each node in the graph 104. Generally, a “feature representation” for a node can characterize any appropriate aspect of the element represented by the node. For example, for a graph that represents a molecule, the initial feature representation 102 for a node can include, e.g., a spatial position (e.g., x, y, and z coordinates) of an atom in the molecule that is represented by the node. In some implementations, the initial feature representation 102 for a node can include an ordered collection of features of the element represented by the node. For example, the initial feature representation for the node can include the spatial position of an atom in the molecule represented by the node (e.g., represented as a vector in [symbol] ³), and a type of atom represented by the node from a set of possible atom types (e.g., including one or more of: carbon, oxygen, nitrogen, etc.).

For one or more of the nodes, the noise engine 160 can generate the final feature representation 112 by modifying (at least a portion of) the initial feature representation 102 using respective noise, e.g., Gaussian noise. In some implementations, the noise engine 160 can randomly sample respective noise values for each of one or more nodes from a distribution, e.g. a Gaussian distribution, and add the respective noise values to the respective initial feature representation 102 for the node to generate the final feature representation 112. For the remaining nodes in the graph 104, the final feature representation can be the same as the initial feature representation. As a particular example, the noise engine 160 can modify the initial feature representation 102 for some, or all, of the nodes in the graph 102 as follows:

\tilde{v}ᵢ=vᵢ+σᵢ (1)

where vᵢ is the initial feature representation for node i, σᵢ is the noise for node i, and \tilde{v}ᵢ is the final feature representation for node i. As a particular example, if the graph 104 represents a molecule, each node in the graph 104 represents an atom in the molecule, and the initial feature representation 102 for a node in the graph 104 includes: (i) a spatial position of the atom represented by the node, and (ii) a type of the atom represented by the node, then the noise engine 160 can generate the final feature representation 112 for the node by adding (or otherwise combining) noise with the features representing the spatial position of the atom represented by the node. That is, for each node in the graph, the noise engine 160 can perturb the features representing the spatial position of the corresponding atom using noise such that the final feature representation for the node defines a perturbed spatial position for the corresponding atom. The respective initial feature representation for each node can further include, e.g., a feature defining the type of the atom represented by the node, and the noise engine 160 can optionally refrain from combining (e.g., adding) noise to the feature representing the atom type.

In some implementations, the noise engine 160 can generate the final feature representation 112 for a node by scaling the initial feature representation 102 for the node using respective noise, e.g., a noise value sampled from the Gaussian distribution. In some implementations, the noise engine 160 can modify the initial feature representation 102 for a node using noise that has the same dimensionality as the initial feature representation 102. For example, if the initial feature representation is an N-dimensional vector, then the noise can also be an N-dimensional vector. Generally, the noise engine 160 can generate the final feature representation 112 for a node in the graph 104 using respective noise in any appropriate manner.

In implementations where the initial feature representations 102 are modified for only some of the nodes in the graph 104 (e.g., not all nodes in the graph 104), the noise engine 160 can randomly select the nodes in the graph 104 for which the initial feature representations 102 are modified.

The graph neural network 150 can include: (i) an encoder 110, (ii) an updater 120, and (iii) a decoder 130, each of which is described in more detail next.

The noise engine 160 can provide the final feature representations 112 for the nodes in the graph 104 to the encoder 110. The encoder 110 can be configured to generate data defining the graph 104 using the respective final feature representations 112 for the nodes. For example, the encoder 110 can assign a respective node in the graph 104 for each element in the system represented by the graph 104. Then, the encoder 110 can instantiate edges between pairs of nodes in the graph 104. Generally, the encoder 110 can instantiate edges between pairs of nodes in the graph 104 in any appropriate manner.

In some implementations, the encoder 110 can instantiate edges between pairs of nodes in the graph 104 by determining, for each pair of nodes, a respective distance between the final feature representations of these nodes. Then, the encoder 110 can determine that each pair of nodes corresponding to a distance that is less than a predefined threshold are connected by an edge in the graph 104. The threshold distance can be any appropriate numerical value. In some implementations, the encoder 110 can instantiate edges between pairs of nodes in the graph 104 based on the type of system being represented by the graph 104.

As a particular example, if the graph 104 represents a molecule, and each node in the graph 104 represents an atom in the molecule, then the encoder 110 can assign an edge in the graph 104 between a pair of nodes that corresponds to a bond between the atoms in the molecule represented by the pair of nodes. In some implementations, the distance between the final feature representations can characterize local interactions between atoms represented by the nodes. For example, the threshold distance can represent a connectivity radius (R), such that the edges connecting pairs of nodes within the connectivity radius represent local interactions of neighboring atoms in the molecule. The search for neighboring nodes in the graph 104 can be performed via any appropriate search algorithm, e.g., a kd-tree algorithm.

In addition to assigning nodes and instantiating edges, the encoder 110 can generate a respective node embedding for each node in the graph 104. Generally, an “embedding” of an entity can refer to a representation of the entity as an ordered collection of numerical values, e.g., a vector or matrix of numerical values.

The encoder 110 can generate the node embedding for each node by using a node embedding sub-network. The node embedding sub-network of the encoder 110 can process the final feature representation 112 for each node in the graph 104 and generate a node embedding for each node in the graph 102. As a particular example, if the graph 104 represents a molecule, the node embedding sub-network can generate the node embedding for the node based on, e.g., a type of atom represented by the node. As another particular example, the node embedding sub-network can generate the node embedding based on whether the atom is a part of an adsorbate or a catalyst, e.g., the node embedding can include 1 for the adsorbate and 0 for the catalyst.

In some implementations, in addition to generating the node embedding for each node in the graph 104, the encoder 110 can generate an edge embedding for each edge in the graph 104 using an edge embedding sub-network of the encoder 110. For example, the edge embedding sub-network of the encoder 110 can process the final feature representations 112 for the nodes in the graph 104 and generate the edge embedding for each edge in the graph 104 based at least in part on a difference between the respective final feature representations 112 for the nodes connected by the edge. As a particular example, an embedding eₖ for an edge k connecting a pair of nodes can be represented as follows:

$${e}_{k} = c o n c at ( {e}_{R B F , 1}^{′} ( \vert d \vert ) , … , {e}_{RBF , c}^{′} ( \vert d \vert ) , \frac{d}{\vert d \vert} ) \qquad ( 2 )$$

where d is the vector displacement for the edge connecting the pair of nodes, |d| is the distance, e′_{RBF,c}(|d|) is a Radial Bessel basis function defined below by equation (3), and concat represents a concatenation operation.

$${e}_{RBF , c}^{′} ( \vert d \vert ) = \sqrt{\frac{2}{r}} \frac{sin ( \frac{c π}{R} d )}{d} \qquad ( 3 )$$

In this manner, the encoder 110 can generate data defining the graph 104 that includes: (i) a set of nodes, (ii) a set of edges that each connect a respective pair of nodes, (iii) a node embedding for each node and, optionally, (iv) an edge embedding for each edge.

After generating data defining the graph 104, the encoder 110 can provide the data to the updater 120. The updater 120 can update the graph 104 over multiple internal update iterations to generate the final graph 106. “Updating” a graph refers to performing a step of message-passing (e.g., a step of propagation of information) between the nodes and edges included in the graph by, e.g., updating the node and/or edge embeddings for some or all nodes and edges in the graph based on node and/or edge embeddings of neighboring nodes in the graph. The updater 120 can include one or more graph neural network layers, and each graph neural network layer can be configured to receive a current graph and update the current graph in accordance with current parameters of the graph neural network layer. The updater 120 can include any number of graph neural network layers, e.g., 1, 10, 100, or any other appropriate number of graph neural network layers. In some implementations, the updater 120 includes at least 10 graph neural network layers.

Specifically, each graph neural network layer can be configured to update a current node embedding of each node in the graph 104 based on: (i) the current node embedding of the node, and (ii) a respective current node embedding of each of one or more neighbors of the node in the graph 104. A pair of nodes in the graph 104 are “neighboring” nodes if they are connected to each other by an edge. In implementations where the graph 104 additionally includes an edge embedding for each edge, each graph neural network layer can update the node embedding of the node also based on a respective edge embedding of each of one or more edges connected to the node in the graph 104.

As a particular example, at each update iteration, each graph neural network layer can be configured to determine a current message vector mᵤᵥ^{(t+1)} for the edge connecting node u to node v as follows:

mᵤᵥ^{(t+1)}=ψₜ₊₁(hᵤ^{t}, hᵥ^{t}, mᵤᵥ^{(t)}+mᵤᵥ^{(t-1)}) (4)

where hᵤ^{t} is the node embedding of node u at the previous update iteration, hᵥ^{t} is the node embedding of node v at the previous update iteration, mᵤᵥ^{(t)} and mᵤᵥ^{(t-1)} are the message vectors for the edge each determined at a previous respective update iteration, and ψₜ₊₁ is the message function implemented by the graph neural network layer as, e.g., a fully-connected neural network layer (e.g. the same for each edge). After determining the message vector, at each update iteration, the graph neural network layer can update the current node embedding hᵤ^{t} for node u, connected to node v by the edge, as follows:

hᵤ^{(t+1)}=φₜ₊₁(hᵤ^{t}, Σ_{N}_{ᵥ}mᵥᵤ^{(t+1)}, Σ_{N}_{ᵤ}mᵤᵥ^{(t+1)})+hᵤ^{t} (5)

where hᵤ^{(t+1)} is the updated node embedding for the update iteration, the update function φₜ₊₁ is implemented by the graph neural network layer as, e.g., a fully-connected neural network layer (e.g. the same for each node), the first sum is over the total number of neighboring nodes Nᵥ of node v, and the second sum is over the total number of neighboring nodes Nᵤ of node u.

The final update iteration of the updater 120 generates data defining the final graph 106. The final graph 106 can have the same structure as the initial graph 104 (e.g., the final graph 106 can have the same number of nodes and the same number of edges as the initial graph 104), but different node embeddings. In some implementations, the final graph 104 can additionally include different edge embeddings.

The updater 120 can provide data defining the final graph 106 to the decoder 130. The decoder 130 can be configured to process data defining the final graph 106 to generate a de-noising prediction 108 for each of one or more nodes having modified feature representations. Specifically, for each of one or more nodes having modified feature representations, the decoder 130 can process the updated node embedding for the node using one or more neural network layers to generate the respective de-noising prediction 108 for the node. The de-noising prediction 108 can characterize a de-noised feature representation for the node that does not include the noise used to generate the modified feature representation for the node.

In some implementations, the de-noising prediction 108 for the node can predict the noise used to generate the modified feature representation for the node.

In some implementations, the de-noising prediction 108 for the node can predict the initial feature representation for the node. For example, if the graph 104 represents a molecule, then the de-noising prediction 108 for the node can predict the initial spatial position of the atom in the molecule represented by the node before the initial spatial position was modified by using noise.

In some implementations, the de-noising prediction 108 for the node can characterize a target feature representation for the node. A “target feature representation” for a node can characterize any appropriate aspect of the element represented by the node in the graph 104. As a particular example, if the initial feature representation 102 for the node includes a spatial position of an atom in a molecule, then the target feature representation for the node can include a different spatial position of the atom in the molecule, e.g., a spatial position of the atom after atomic relaxation of the molecule. As another example, if the graph represents a social network and each node in the graph represents a respective user in the social network, then a target embedding for each node can characterize, e.g., an amount of time (e.g., in minutes) that the corresponding user interacts with the social network over a designated time period (e.g., one day). In one example, the de-noising prediction 108 for each node can include an output feature representation that is an estimate of the target feature representation for the node. In another example, the de-noising prediction 108 for each node can include a prediction for an incremental feature representation for the node that, if added to the modified feature representation for the node, results in the target feature representation of the node.

Because generating the de-noising predictions 108 requires each node embedding to encode unique information in order to de-noise the feature representation of the node, this can mitigate the effects of “over-smoothing,” e.g., where the node embeddings become nearly identical after being processed through a number of graph neural network layers.

In some implementations, the decoder 130 can process the updated node embeddings of the nodes to generate a task prediction 109. The task prediction can be, e.g., a single output for the input graph 104, or a respective output for each node in the input graph 104. Generally, the task prediction 109 can be any appropriate prediction characterizing one or more of the elements represented by the nodes in the graph 104. The task prediction 109 can be, e.g., a classification prediction or a regression prediction. A classification prediction can include a respective score for each class in a set of possible classes, where the score for a class can define a likelihood that the set of elements represented by the graph 104 are included in the class. A regression prediction can include one or more numerical values, each drawn from a continuous range of values, that characterize the set of elements represented by the graph 104.

In one example, in order to generate the task prediction, the decoder 130 can process (i) the updated node embeddings of the nodes, and (ii) original node embeddings of the nodes prior to being updated using the graph neural network layers. As a particular example, the decoder 130 can generate the task prediction y as follows:

y=W^{Update}Σᵢ₌₁^{|V|}MLP_{Update}(aᵢ^{Update})+b^{Update}+W^{Enc}Σᵢ₌₁^{|V|}MLP_{Enc}(aᵢ^{Enc})+b^{Enc} (6)

where aᵢ^{Update} is the updated node embedding of node i, aᵢ^{Enc} is the original node embedding of node i, |V| is the total number of nodes in the graph, MLP_{Update} and MLP_{Enc} are, e.g., fully-connected neural network layers of the updater and the encoder, respectively, b^{Update} is a bias term of the updater, b^{Enc} is a bias term of the encoder, W^{Update} is a linear neural network layer of the updater, and W^{Enc} is a linear neural network layer of the encoder.

As a particular example, if the graph 104 represents a molecule, then the task prediction 109 can be a prediction of one or more of: an equilibrium energy, an internal energy, or a highest occupied molecular orbital (HOMO) energy of the molecule represented by the graph 104. In some implementations, the decoder 130 can process the updated node embeddings of fewer than all of the nodes to generate the task prediction 109, e.g., in some cases, the decoder 130 can generate the task prediction 109 by processing the updated node embedding of a single node in the graph 104. Examples of task predictions are described in more detail below.

The encoder 110, the updater 120, and the decoder 130, can have any appropriate neural network architecture that enables them to perform their prescribed functions. For example, the encoder 110, the updater 120, and the decoder 130, can have any appropriate neural network layers (e.g., convolutional layers, fully connected layers, recurrent layers, attention layers, graph neural network layers, etc.) in any appropriate numbers (e.g., 2 layers, 5 layers, or 10 layers) and connected in any appropriate configuration (e.g., as a linear sequence of layers).

The system 100 can further include a training engine 140 that can train the neural network 150 using the de-noising predictions 108. The training engine 150 can evaluate an objective function that measures, for one or more of the graph neural network layers of the neural network 150, respective errors in de-noising predictions 180 for the nodes that are based on updated node embeddings generated by the graph neural network layer. More specifically, the graph neural network 150 can generate respective de-noising predictions for the nodes in the graph 104 at each of one of more graph neural network layers, i.e., based on the updated node embeddings generated by the graph neural network layer. In some implementations, the objective function can additionally measure an error in the task prediction 109, e.g., using a cross-entropy error measure, a squared-error measure, or any other appropriate error measure. As a particular example, the objective function [symbol] can be represented as follows:

[symbol]

=λ [symbol] _{de-noising}+ [symbol] ₜₐₛₖ (7)

where [symbol] _{de-noising} measures respective errors in de-noising predictions for the nodes, [symbol] ₜₐₛₖ measures an error in the task prediction, and λ is a weight factor. The weight factor and noise will vary according to the application and may be optimized with hyperparameter sweeps; merely as an example the weight factor may be of order 0.1 and the noise standard deviation of order 0.01.

To optimize the objective function, the training engine 140 can determine gradients of the objective function with respect to the current values of neural network parameters, e.g., using backpropagation techniques. The training engine 140 can then use the gradients to update the current values of the neural network parameters, e.g., using any appropriate gradient descent optimization technique, e.g., an RMSprop or Adam gradient descent optimization technique. Specifically, the training engine 140 can backpropagate gradients of the objective function through neural network parameters of the graph neural network layers.

In some implementations, the training engine 140 can first pre-train the neural network 150 to optimize an objective function based only on the de-noising predictions (e.g., [symbol] _{de-noising}), and then train the neural network 150 to optimize the objective function based on both the de-noising predictions and the task predictions (e.g., [symbol] as defined in equation (7)). Training the neural network 150 to generate de-noising predictions 108 can reduce the likelihood of “over-fitting,” e.g., because the noise added to the feature representations of the nodes in the graph 104 prevents the neural network 150 from memorizing the initial node feature representations.

In some implementations, the training engine 140 can pre-train the neural network 150 to optimize an objective function based on only the de-noising predictions, and then train the neural network 150 to optimize an objective function based on only the task predictions.

In some implementations, the training engine 140 can pre-train the neural network 150 to optimize an objective function based on the de-noising predictions and a first task prediction, and then train the neural network 150 to optimize an objective function based on only a second task prediction. The second task prediction can be different than the first task prediction. For instance, the first task prediction can include predicting HOMO energies of molecules, while the second task prediction can include predicting equilibrium energies of molecules.

Optionally, after pre-training the neural network 150 to optimize an objective function based on the de-noising predictions, the training engine 140 can “freeze” some of the parameters of the neural network, and then train only the unfrozen parameters of the neural network on an objective function based on a task prediction. (Freezing a parameter of a neural network can refer to designating the current value of the parameter as a fixed, static value that is not modified further during training). For example, the training engine 140 can pre-train the neural network to optimize an objective function based on the de-noising predictions, freeze the parameters of the encoder 110 and the updater 120, and then train only the parameters of the decoder on an objective function based on a task prediction.

Example applications of the system 100, and of the trained graph neural network 150, are described in more detail next. In general using the trained graph neural network 150 involves obtaining feature representations for the nodes; generating the data defining the graph 104 using the feature representations for the nodes; and processing the data defining graph 104 using the graph neural network 150 to generate a respective updated node embedding for each node. The output from the graph neural network may then comprise, depending on the application, one or more of: features decoded from the updated node embeddings of the graph 104; the de-noising prediction 108; and the task prediction 109.

In some implementations, the graph 104 can represent one or more molecules; here a “molecule” includes e.g. a large slab of atoms such as a surface of a catalyst. Then each node in the graph 104 can represent a respective atom in the molecule(s). In general the feature representation of a node defines the type of atom represented by the node. It may include other features such as atomic number, whether the atom is an electron or proton donor or acceptor, whether the atom is part of an aromatic system, a degree of hybridization e.g. for a carbon atom, and where hydrogen atoms are not explicitly represented, a number of hydrogens attached to the atom. In some implementations the feature representation of a node may include a (3D) spatial position of an atom in the molecule. In some implementations the feature representation a node does not include a (3D) spatial position of an atom in the molecule, i.e. the molecule may be defined by bonds and atom types. Where the representation does not include a spatial position noise may be added by randomly changing one or more features or a node (and optionally also one or more features of an edge); a de-noising prediction may then comprise a reconstruction of the features. In some implementations, e.g. where two or more interacting entities are modelled, a node feature may indicate to which of the entities the atom belongs. Thus in general the feature representation of the nodes define the structure and nature (e.g. types of the atoms) of the molecule(s).

In some implementations the neural network is trained to identify a resulting structure of the atoms from an initial structure of the atoms in the molecule(s). The structure may be decoded from the node embeddings, e.g. as (3D) spatial positions of the atoms decoded from the node embeddings, or as bonds and atom types; for example in some implementations it may be derived from the respective de-noising predictions for the nodes. Also or instead the neural network is trained to generate a task prediction where the task prediction can characterize one or more predicted properties of the molecule, e.g., the equilibrium energy of the molecule, the energy required to break up the molecule, or the charge of the molecule. For example the task is to predict one or more characteristics of the molecule(s) such as: a binding state prediction e.g. a measure of how tightly the atoms are bound, such as a measure of an energy needed to break apart one or more of the molecules, or a measure of bond angles or lengths; or a HOMO or LUMO energy of one or more of the molecules; or a characteristic of a distribution of electrons in the molecule(s) such as size, charge, dipole moment, or static polarizability.

Local, random distortions of the geometry of a molecule or molecules at a local energy minimum are almost certainly higher energy configurations. Thus in implementations where the de-noising prediction for a node predicts the initial spatial position of an atom before it was modified by using noise, the neural network is implicitly trained to determine an equilibrium or relaxed structure from the initial structure.

As previously mentioned, the trained graph neural network 150 can be used by using features decoded from the node embeddings of the graph 104, or the de-noising prediction, or the task prediction, depending on the application.

One example application involves using the trained graph neural network 150 to obtain a catalyst molecule or a molecule that interacts with a catalyst. In this application the feature representations for the nodes comprise features defining the structure and nature of the catalyst molecule or a molecule that interacts with a catalyst. The output from the graph neural network may then comprise, e.g., features decoded from the updated node embeddings of the graph or from the de-noising prediction representing a resulting structure when the molecules interact; and/or a task prediction characterizing a resulting state of the molecules e.g. an equilibrium energy of the molecules, or a change in energy resulting from the interaction, or an energy required to break apart the molecules. The resulting structure or the prediction characterizing the resulting state of the molecules may be used to obtain the catalyst molecule or the molecule that interacts with a catalyst, e.g. by screening a plurality of candidate molecules. The screening may be to identify those that interact in a desirable manner, e.g. particularly strongly; or to screen out unsuitable molecules; or to identify a catalyst molecule that interacts with multiple other molecules, or a molecule that interacts with multiple different catalyst molecules (which can be either useful or unwanted).

The screening process may, e.g. involve determining a score for each of a plurality of candidate catalyst molecules and/or candidate molecules that interacts with the catalyst using the output from the graph neural network; and selecting one or more of the candidates using the score. The method may further involve making a catalyst molecule or a molecule that interacts with a catalyst that is obtained by the method; and optionally testing the interaction in the real-world.

In a particular example of this application the catalyst molecule comprises an enzyme or the receptor part of an enzyme, and the molecule that interacts with the catalyst is a ligand of the enzyme, e.g. an agonist or antagonist of the receptor or enzyme. The ligand may be e.g. a drug or a ligand of an industrial enzyme. One or both of the molecules may comprise a protein molecule.

A further related application involves using the trained graph neural network 150 to identify a drug molecule that inhibits replication of a pathogen, i.e. to obtain a drug molecule that interacts with a pathogen molecule. The pathogen molecule is a molecule that is associated with the pathogen, where replication of a pathogen is inhibited when the drug molecule interacts with the pathogen molecule. Thus in the above-described method the pathogen molecule is used in place of the catalyst molecule. Thus the feature representations for the nodes may then comprise features defining the molecules and the output from the graph neural network is used to screen candidate drug molecules and or pathogen molecules to obtain the drug molecule. The method may also involve making, and optionally testing the drug molecule against the pathogen in the real world.

Another example application involves using the trained graph neural network 150 to determine the reaction mechanism of a chemical reaction to make a product that involves two or more molecules interacting. One or more of the molecules may then be modified to modify the reaction mechanism e.g. to increase a speed of the reaction or product yield. The reaction mechanism may then be used to make the product. The feature representations for the nodes may then comprise features defining the molecules. The output from the graph neural network may comprise, e.g., features decoded from the updated node embeddings of the graph or from the de-noising prediction representing a resulting structure when the molecules interact; and/or a task prediction characterizing a resulting state of the molecules. For example the output may predict one or more of an energetic state, a binding state, and a conformation of one or more transition states of the molecules along a reaction coordinate.

In the above applications the feature representations for the nodes may then comprise features determined from one or more measurements made on real-world molecules, e.g. using electron microscopy to characterize the structure or nature of the molecule(s). The features obtained in this way may then be processed by the trained graph neural network to obtain the graph neural network output, e.g. the task prediction output to characterize one or more properties of the molecule(s), e.g. the equilibrium energy, the binding state, a measure of bond angles or lengths; a HOMO or LUMO energy, or a size, charge, dipole moment, or static polarizability.

Some example training datasets that can be used to train the graph neural network 150 to perform the above tasks are: The OC20 dataset (Chanussot et al., “The Open Catalyst 2020 (OC20) Dataset and Community Challenges”, ACS Catalysis, 6059-6072, 2020, arXiv:2010.09990); the QM9 dataset, Ramakrishnan et al., “Quantum chemistry structures and properties of 134 kilo molecules”, Sci Data 1, 140022 (2014); the OGBG-PCQM4M dataset from Open Graph Benchmark, Hu et al., “Open Graph Benchmark: Datasets for Machine Learning on Graphs”, arXiv:2005.00687; and OGBG-MOLPCBA, also from the Open Graph Benchmark.

In some implementations, the graph 104 can represent a physical system, each node in the graph 104 can represent a respective object in the physical system, and the task prediction can characterize a respective predicted future state of one or more objects in the physical system, e.g., a respective position and/or velocity of each of one or more objects in the physical system at a future time point.

One example application involves using the trained graph neural network 150 to predict a state of or control the physical system. The feature representations for the nodes may comprise features determined from the objects. Such features may comprise a mass, or moment of inertia, position, orientation, linear or angular speed, or acceleration of an object; edges may represent connected or interacting objects e.g. objects connected by a joint. The output from the graph neural network e.g. features decoded from the updated node embeddings, de-noising prediction 108, or the task prediction may define e.g. a prediction of a future state of the objects in the physical system for a single time step or for a rollout over multiple time steps. The output may be used to provide action control signals for controlling the objects dependent upon the future state. For example the trained graph neural network 150 may be included in a Model Predictive Control (MPC) system to predict a state or trajectory of the physical system for use by a control algorithm in controlling the physical system, e.g. to maximize a reward or minimize a cost predicted from the future state.

In some implementations, the graph 104 can represent a point cloud (e.g., generated by a lidar or radar sensor), each node in the graph 104 can represent a respective point in the point cloud, and the task prediction can predict a class of object represented by the point cloud.

In some implementations, the graph 104 can represent a portion of text, each node in the graph 104 can represent a respective word in the portion of text, and the task prediction can predict, e.g., a sentiment expressed in the portion of text, e.g., positive, negative, or neutral.

In some implementations, the graph 104 can represent an image, each node in the graph 104 can represent a respective portion of the image (e.g., a pixel or a region of the image), and the task prediction can characterize, e.g., a class of object depicted in the image.

In some implementations, the graph 104 can represent an environment in the vicinity of a partially- or fully-autonomous vehicle, each node in the graph can represent a respective agent in the environment (e.g., a pedestrian, bicyclist, vehicle, etc.) or an element of the environment (e.g., traffic lights, traffic signs, road lanes, etc.), and the task prediction can predict, e.g., a respective future trajectory of one or more of the agents represented by nodes in the graph. For example, the prediction output can characterize a respective likelihood that a vehicle agent represented by a node in the graph will make one or more possible driving decisions, e.g., going straight, changing lanes, turning left, or turning right. In this example, to predict a future trajectory of an agent represented by a node in the graph, the system can process the update node embedding for only the node representing the agent, i.e., without processing the updated node embeddings for the other nodes in the graph. Edges of the graph may represent, e.g. physical proximity or connectedness of the agents or elements; connectedness may be defined as the existence of route such as a road or pathway connecting the agents or elements. For example the trained graph neural network may be used to control a mechanical agent in a real-world environment. The trained graph neural network may process feature representations for the nodes that comprise features representing the other agents or elements of the environment, e.g. for each agent or element a type of the other agent or element, and a position, configuration, orientation, linear or angular speed, or acceleration of the other agent or element to generate the graph neural network output for controlling the agent.

In some implementations, the graph 104 can represent a social network (e.g., on a social media platform), each node in the graph can represent a respective person in the social network, each edge in the graph can represent, e.g., a relationship between two corresponding people in the social network (e.g., a “follower” or “friend” relationship), and the task prediction can predict, e.g., which people in the social network are likely to perform a certain action in the future (e.g., purchase a product or attend an event).

In some implementations, the graph 104 can represent a road network, each node in the graph can represent a route segment in the road network, each edge in the graph can represent that two corresponding route segments are connected in the road network, and the task prediction can predict, e.g., a time required to traverse a specified path through the road network, or an amount of traffic on a specified path through the road network.

In some implementations, the graph 104 can be a computational graph that represents, e.g., computational operations performed by a neural network model, each node in the graph can represent a group of one or more related computations (e.g., operations performed by a group of one or more neural network layers), and each edge in the graph can represent that an output of one group of computations is provided as an input to another group of computations. In these implementations, the task prediction can predict, e.g., a respective computing unit (i.e., from a set of available computing units) that should perform the operations corresponding to each node in the graph, e.g., to minimize a time required to perform the operations defined by the graph. Each computing unit can be, e.g., a respective thread, central processing unit (CPU), or graphics processing unit (GPU). Thus the trained graph neural network may be used to perform a task that assigns computational operations to physical or logical computing units. The trained graph neural network may process feature representations for the nodes that comprise features representing groups of computations to generate the graph neural network output (e.g. features decoded from the updated node embeddings, the de-noising prediction, or the task prediction) to identify a respective computing unit that should perform the operations corresponding to each node in the graph.

In some implementations, as described above, the graph 104 can represent a protein, each node in the graph can represent a respective amino acid in the amino acid sequence of the protein, and each edge in the graph can represent that two corresponding amino acids in the protein are separated by less than a threshold distance (e.g., 8 Angstroms) in a structure of the protein. In these implementations, the task prediction can predict, e.g., a stability of the protein, or a function of the protein.

In some implementations, the graph 104 can represent a knowledge base, each node in the graph can represent a respective entity in the knowledge base, and each edge in the graph can represent a relationship between two corresponding entities in the knowledge base. In these implementations, the task prediction can predict, e.g., missing features associated with one or more entities in the knowledge base.

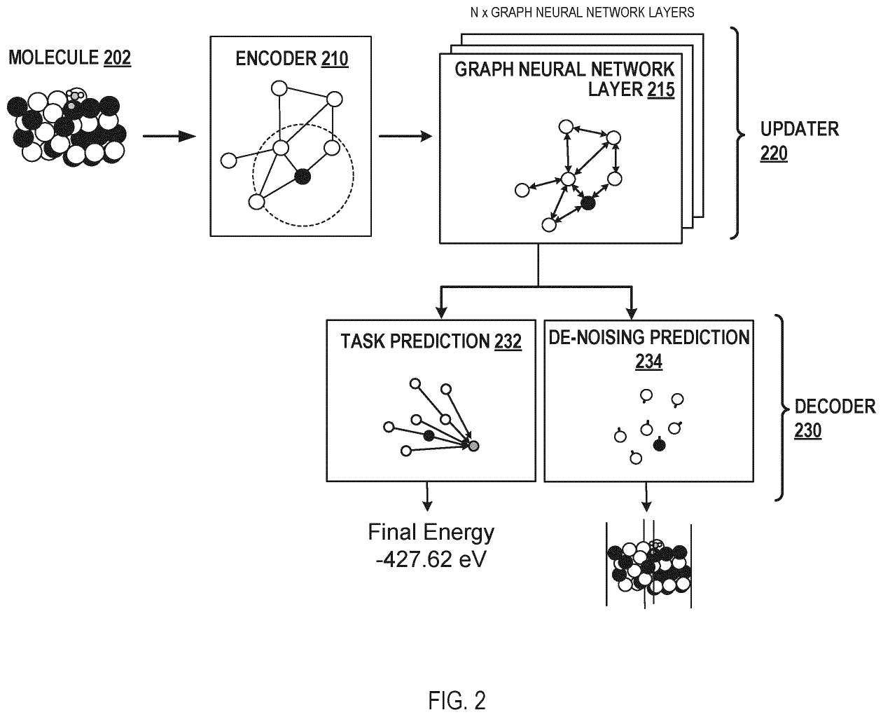

FIG. 2 illustrates an example of operations that can be performed by the training system 100 in FIG. 1. The system 100 can train the graph neural network 150 by using the neural network 150 to generate de-noising predictions 234. In some implementations, the system 100 can additionally train the neural network 150 by using the neural network 150 to generate task predictions 232.

As described above with reference to FIG. 1, the system 100 can train the neural network by using an objective function defined in equation (7). At any training iteration, the objective function can include terms measuring the error in: (i) the de-noising predictions (e.g., [symbol] _{de-noising}), (ii) the task prediction (e.g., [symbol] ₜₐₛₖ), or (iii) both. In some implementations, the system 100 can first pre-train the neural network 150 to optimize the objective function based only on the de-noising predictions (e.g., [symbol] _{de-noising}), and then train the neural network 150 to optimize the objective function based on both the de-noising predictions and the task predictions.

The system can include a noise engine that can be configured to generate final feature representations for nodes in a graph. In some implementations, the graph can represent a molecule 202, and each node in the graph can represent a respective atom in the molecule 202. In such cases, the noise engine can process an initial feature representation for each node in the graph, where the initial feature representation for a node represents an initial spatial position of a corresponding atom in the molecule 202. The noise engine can generate final feature representations for the nodes, where the final feature representations for some, or all, of the nodes are modified feature representations that are generating using respective noise. For example, the noise engine can generate the final feature representation for a node by adding noise to the initial spatial position of the atom in the molecule 202 represented by the node.

The neural network 150 can include: (i) an encoder 210, (ii) an updater 220, and (iii) a decoder 230.

The noise engine can provide final feature representations for the nodes in the graph to the encoder 210. The encoder 210 can be configured to generate data defining the graph. For example, the encoder 210 include a node embeddings sub-network that can generate a node embedding for each node based on, e.g., the final feature representations for each node.

After generating data defining the graph, the encoder 210 can provide the data defining the graph to the updater 220. The updater can include one or more graph neural network layers, e.g., N graph neural network layers. Each graph neural network layer can be configured to update a current node embedding of one or more nodes in the graph based on the current node embedding of the node and respective current node embedding of each of one or more neighbors of the node in the graph. For example, as illustrated in FIG. 2, the graph neural network layer 215 can update the node embedding of the node shown by a filled circle based on the current node embeddings of the neighboring nodes.

The decoder 230 can be configured to process, for each of one or more of the nodes having modified feature representations, the updated node embedding of the node to generate respective de-noising prediction 234 for the node. The de-noising prediction 234 can characterize a de-noised feature representation for the node that does not include the noise used to generate the modified feature representation of the node. As described above with reference to FIG. 1, the de-noising prediction 234 can predict the noise used to generate the modified feature representation for the node. In some implementations, the de-noising prediction 234 can predict the initial feature representation for the node, e.g., an initial spatial position of the atom in the molecule 202 represented by the node, before it was corrupted with noise.

In some implementations, the de-noising prediction 234 for the node can characterize a target feature representation of the node. For example, as illustrated in FIG. 2, the target feature representation can specify a final spatial position of the atom in the molecule 202 represented by the node after atomic relaxation. In this manner, the system 100 can map initial spatial positions of atoms in the molecule 202 (e.g., specified by initial feature representations for the nodes), to final spatial positions of atoms in the molecule 202.

In some implementations, the decoder 230 can generate a task prediction 232. Generally, the task prediction 232 can be any appropriate prediction characterizing one or more of the elements represented by the nodes in the graph. As illustrated in FIG. 2, the task prediction 232 can be, e.g., an equilibrium energy of the molecule 202 after atomic relaxation.

Training the neural network 150 to generate de-noising predictions 234 can encourage the neural network 150 to implicitly learn the distribution of “real” graphs, i.e., with unmodified node feature representations, and the neural network 150 can leverage this implicit knowledge to achieve higher accuracy on task predictions 232.

An example process for using the training system 100 to train the neural network 150 is described in more detail next.

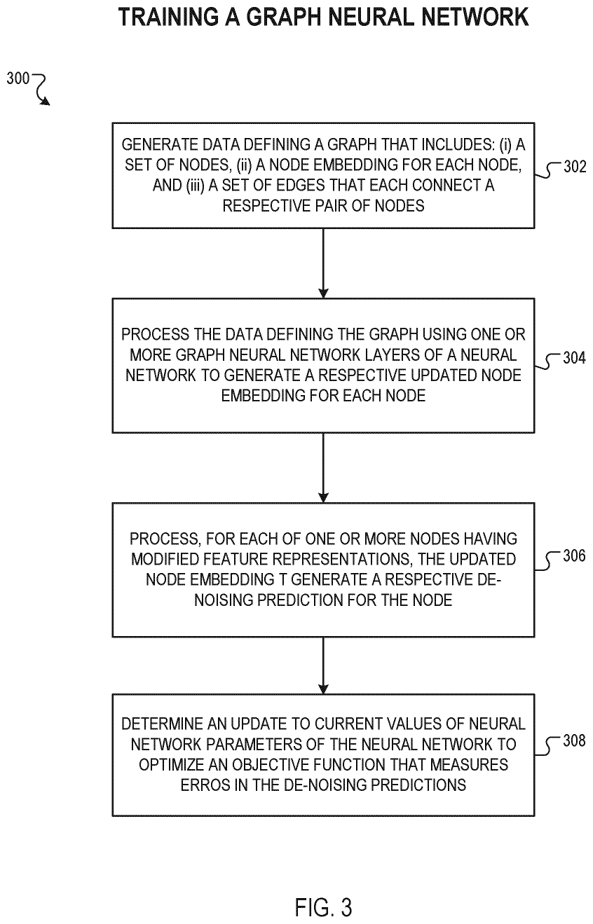

FIG. 3 is a flow diagram of an example process 300 for using a training system to train a graph neural network. For convenience, the process 300 will be described as being performed by a system of one or more computers located in one or more locations. For example, a training system, e.g., the training system 100 of FIG. 1, appropriately programmed in accordance with this specification, can perform the process 300.

The system generates data defining a graph that includes: (i) a set of nodes, (ii) a node embedding for each node, and (iii) a set of edges that each connect a respective pair of nodes (302). As described above with reference to FIG. 1, the system can generate data defining the graph based on feature representations for each node. For example, the system can obtain a respective initial feature representation for each node and generate a respective final feature representation for each node. For each of one or more of the nodes, the respective final feature representation can be a modified feature representation that is generated from the respective feature representation for the node using respective noise, e.g., by adding the respective noise to the respective feature representation for the node, as defined by equation (1).

The system can generate the data defining the graph using the respective final feature representations of the nodes. For example, the system can determine, for each pair of nodes including a first node and a second node, a respective distance between the final feature representation for the first node and the final feature representation for the second node. Then, the system can determine that each pair of nodes corresponding to a distance that is less than a predefined threshold are connected by an edge in the graph. As illustrated in FIG. 2, the graph can represent a molecule and each node in the graph can represent a respective atom in the molecule. In this case, the initial feature representation for each node can represent e.g. an initial spatial position of a corresponding atom in the molecule. The system can generate data defining the graph by generating a node embedding for each node based on a type of atom represented by the node. Generally, the graph can represent any appropriate physical system.

In some implementations, the graph can further include a respective edge embedding for each edge. In such cases, the system can generate the graph by generating an edge embedding for each edge in the graph based at least in part on a difference between the respective final feature representations of the nodes connected by the edge, e.g., as defined by equation (2) and equation (3) above.

The system processes the data defining the graph using one or more graph neural network layers of the neural network to generate a respective updated node embedding of each node (304). In some implementations, the neural network includes at least 10 graph neural network layers. Each graph neural network layer can be configured to update a current graph.

For example, as described above with reference to FIG. 1, each neural network layer can receive the current graph and update the current graph in accordance with current neural network parameter values of the graph neural network layer. This can include, for example, updating a current node embedding of each of one or more nodes in the graph based on: (i) the current node embedding of the node, and (ii) a respective current node embedding of each of one or more neighbors of the node in the graph, e.g., as defined by equation (4) and equation (5) above. In some implementations, the current graph can further include an edge embedding for each edge. In such cases, each neural network layer can update the node embedding of the node based at least in part on a respective edge embedding of each of one or more edges connected to the node.

The system processes, for each of one or more of the nodes having modified feature representations, the updated node embedding of the node to generate a respective de-noising prediction for the node (306). For example, the system can process the updated node embedding of the node using one or more neural network layers to generate the respective de-noising prediction for the node. The de-noising prediction can characterize a de-noised feature representation for the node that does not include the noise used to generate the modified feature representation of the node.

In one example, the de-noising prediction can predict the noise used to generate the modified feature representation of the node. In another example, the de-noising prediction can predict the initial feature representation of the node, e.g., the initial spatial position of an atom in a molecule before it was modified by using noise. In yet another example, the de-noising prediction can characterize a target feature representation of the node, e.g., a new position of the atom in the molecule after atomic relaxation. In some implementations, the target feature representation for the node can be an incremental feature representation for the node that, if added to the modified feature representation for the node, results in the target feature representation of the node.

The system determines an update to current values of neural network parameters of the neural network to optimize an objective function that measures errors in the de-noising predictions for the nodes (308). For example, the system can backpropagate gradients of the objective function through neural network parameters of the graph neural network layers. The objective function can measure, for each of multiple graph neural network layers of the neural network, respective errors in de-noising predictions for the nodes that are based on updated node embeddings generated by the graph neural network layer.

In some implementations, the system can process the updated node embeddings of the nodes to generate a task prediction, where the objective function also measures an error in the task prediction, e.g., as defined in equation (7). In general the error in the task prediction may be determined using a set of training data appropriate to the task, i.e. the system may be trained using supervised learning. In such cases, the system can process both: (i) the updated node embeddings of the nodes, and (ii) original node embeddings of the nodes prior to being updated using the graph neural network layers, to generate the task prediction. As a particular example, the graph can represent a molecule and the task prediction can be a prediction of an equilibrium energy of the molecule, e.g., as illustrated in FIG. 2.

Example experimental results achieved using the system for training the neural network are described in more detail next.

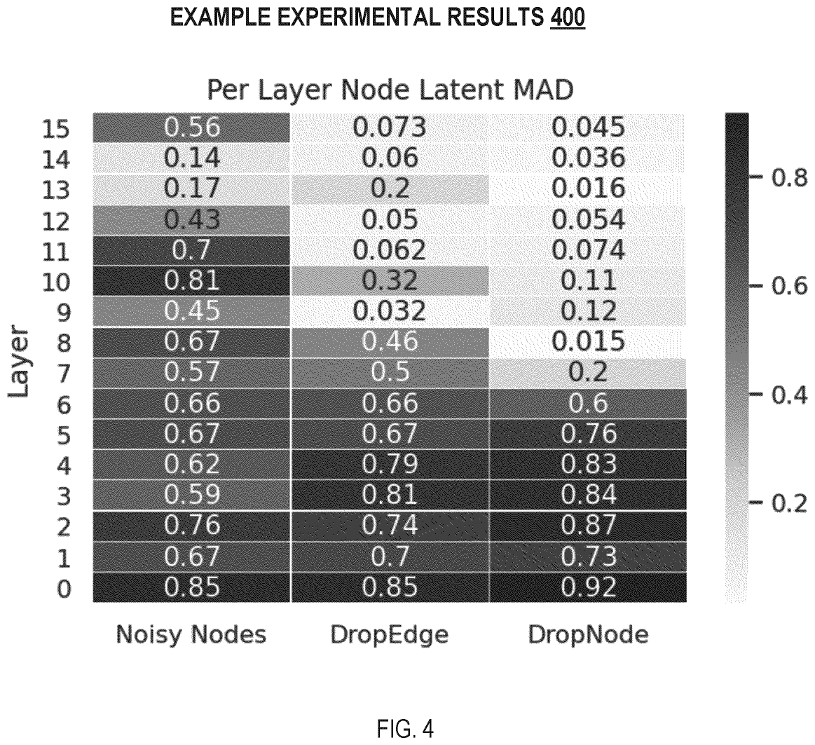

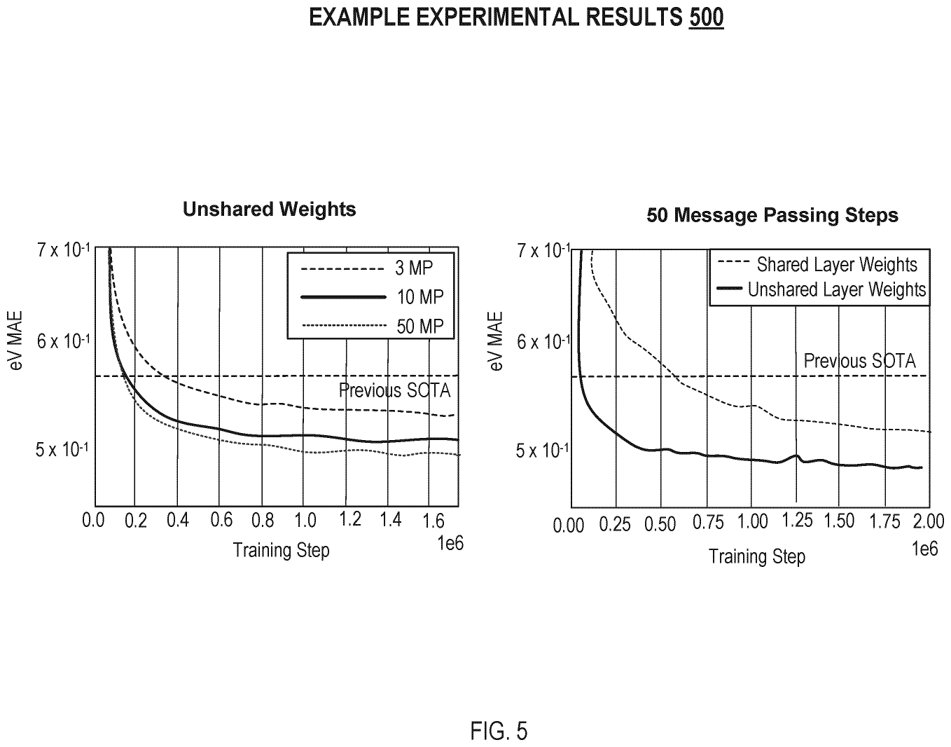

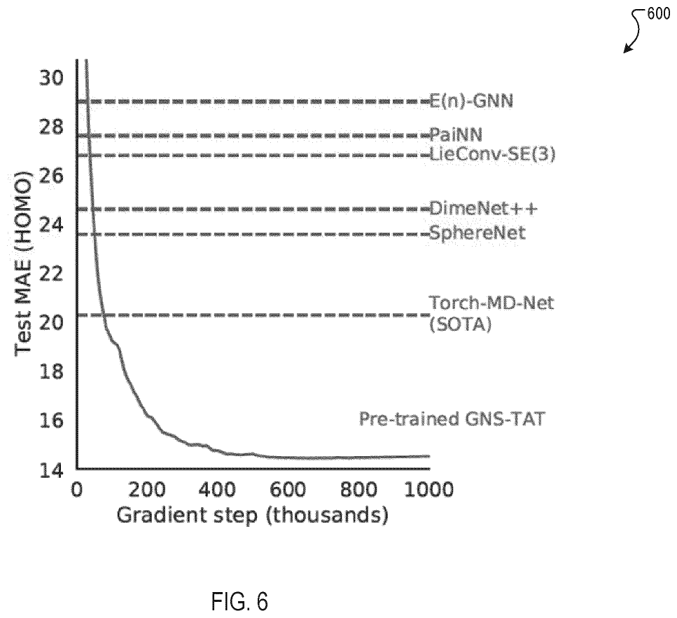

FIG. 4 illustrates example experimental results 400 achieved using the system 100 for training a neural network described above with reference to FIG. 1 and FIG. 2.

The system 100 can train the neural network by using the neural network to generate de-noising predictions. As described above, generating de-noising predictions requires each node embedding to encode unique information in order to de-noise the feature representation of the node, which can mitigate the effects of “over-smoothing,” e.g., where the node embeddings become nearly identical after being processed through a number of graph neural network layers. In FIG. 4, “MAD” is a measure of diversity of node embeddings that can quantify “over-smoothing,” where a higher number indicates a higher level of diversity of node embeddings. As illustrated in FIG. 4, the system described in this specification is able to maintain a higher level of node embedding diversity throughout the neural network, when compared to other available techniques (e.g., “DropEdge” and “DropNode”). This is particularly evident at the neural network layer 15 where the measure of diversity of node embeddings of the system described in this specification is much higher. Therefore, the system described in this specification can outperform other available systems at mitigating the effects of “over-smoothing.”

FIG. 5 illustrates example experimental results 500 achieved using the system 100 for training a neural network described above with reference to FIG. 1 and FIG. 2. In FIG. 5, “Previous SOTA” refers to state-of-the-art performance achieved using other available systems, and “ev MAE” refers to prediction error on the task prediction.

The left-hand side graph shows that even after 3 message-passing steps (e.g., with 3 graph neural network layers in the neural network), the system described in this specification surpasses state-of-the-art performance achieved using other available systems. The right-hand side graph shows the state-of-the-art performance can be surpassed by the system described in this specification even with a smaller number of neural network parameters (e.g., with shared weights between graph neural network layers).

FIG. 6 illustrates example experimental results comparing the performance of various neural networks on the task of predicting the HOMO energy of molecules. In particular, the horizontal axis of the graph 600 represents a number of gradient steps used to train each neural network, and the vertical axis of the graph 600 represents the prediction accuracy of each neural network. The best-performing neural network (labeled “Pre-trained GNS-TAT” in FIG. 6) is a graph neural network that is pre-trained to optimize an objective function based on de-noising predictions (as described in this specification) prior to being trained to perform the HOMO energy prediction task. These experimental results thus illustrate advantages that can be achieved by pre-training a graph neural network to optimize an objective function based on de-noising predictions.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method for training a neural network that includes one or more graph neural network layers, the method comprising:
generating data defining a graph that comprises: (i) a set of nodes, (ii) a node embedding for each node, and (iii) a set of edges that each connect a respective pair of nodes, comprising:
obtaining a respective initial feature representation for each node;
generating a respective final feature representation for each node, wherein, for each of one or more of the nodes, the respective final feature representation is a modified feature representation that is generated from the respective feature representation for the node using respective noise; and
generating the data defining the graph using the respective final feature representations of the nodes;
processing the data defining the graph using one or more of the graph neural network layers of the neural network to generate a respective updated node embedding of each node;
processing, for each of one or more of the nodes having modified feature representations, the updated node embedding of the node to generate a respective de-noising prediction for the node that characterizes a de-noised feature representation for the node that does not include the noise used to generate the modified feature representation of the node; and
determining an update to current values of neural network parameters of the neural network to optimize an objective function that measures errors in the de-noising predictions for the nodes.

2. The method of claim 1, wherein for each of one or more of the nodes having modified feature representations, the respective de-noising prediction for the node predicts the noise used to generate the modified feature representation of the node.

3. The method of claim 1, wherein for each of one or more of the nodes having modified feature representations, the respective de-noising prediction for the node predicts the respective initial feature representation of the node.

4. The method of claim 1, wherein for each of one or more of the nodes having modified feature representations, the respective de-noising prediction for the node characterizes a target feature representation of the node.

5. The method of claim 4, wherein for each of one or more of the nodes having modified feature representations, the respective de-noising prediction for the node predicts an incremental feature representation for the node that, if added to the modified feature representation for the node, results in the target feature representation of the node.

6. The method of claim 1, further comprising processing the updated node embeddings of the nodes to generate a task prediction, wherein the objective function also measures an error in the task prediction.

7. The method of claim 6, wherein both: (i) the updated node embeddings of the nodes, and (ii) original node embeddings of the nodes prior to being updated using the graph neural network layers, are processed to generate the task prediction.

8. The method of claim 6, wherein the graph represents a molecule and the task prediction is a prediction of an equilibrium energy of the molecule.

9. The method of claim 1, wherein the objective function measures, for each of a plurality of graph neural network layers of the neural network, respective errors in de-noising predictions for the nodes that are based on updated node embeddings generated by the graph neural network layer.

10. The method of claim 1, wherein for each of one or more of the nodes having modified feature representations, processing the updated node embedding of the node to generate the respective de-noising prediction for the node comprises:
processing the updated node embedding of the node using one or more neural network layers to generate the respective de-noising prediction for the node.

11. The method of claim 1, wherein determining the update to the current values of the neural network parameters of the neural network to optimize the objective function comprises:
backpropagating gradients of the objective function through neural network parameters of the graph neural network layers.

12. The method of claim 1, wherein for each of one or more of the nodes, the respective final feature representation for the node is generated by adding the respective noise to the respective feature representation for the node.

13. The method of claim 1, wherein generating the data defining the graph using the respective final feature representations of the nodes comprises:
determining, for each pair of nodes comprising a first node and a second node, a respective distance between the final feature representation for the first node and the final feature representation for the second node; and
determining that each pair of nodes corresponding to a distance that is less than a predefined threshold are connected by an edge in the graph.

14. The method of claim 1, wherein the graph further comprises a respective edge embedding for each edge.

15. The method of claim 14, wherein generating the data defining the graph comprises:
generating an edge embedding for each edge in the graph based at least in part on a difference between the respective final feature representations of the nodes connected by the edge.

16. The method of claim 1, wherein the graph represents a molecule, each node in the graph represents a respective atom in the molecule, and generating the data defining the graph comprises:
generating a node embedding for each node based on a type of atom represented by the node.

17. The method of claim 1, wherein the neural network includes at least 10 graph neural network layers.

18. The method of claim 1, wherein each graph neural network layer of the graph neural network is configured to:
receive a current graph; and
update the current graph in accordance with current neural network parameter values of the graph neural network layer, comprising:
updating a current node embedding of each of one or more nodes in the graph based on: (i) the current node embedding of the node, and (ii) a respective current node embedding of each of one or more neighbors of the node in the graph.

19. (canceled)

20. (canceled)

21. One or more non-transitory computer storage media storing instructions that when executed by one or more computers cause the one or more computers to perform operations for training a neural network that includes one or more graph neural network layers, the operations comprising:
generating data defining a graph that comprises: (i) a set of nodes, (ii) a node embedding for each node, and (iii) a set of edges that each connect a respective pair of nodes, comprising:
obtaining a respective initial feature representation for each node;
generating a respective final feature representation for each node, wherein, for each of one or more of the nodes, the respective final feature representation is a modified feature representation that is generated from the respective feature representation for the node using respective noise; and
generating the data defining the graph using the respective final feature representations of the nodes;
processing the data defining the graph using one or more of the graph neural network layers of the neural network to generate a respective updated node embedding of each node;
processing, for each of one or more of the nodes having modified feature representations, the updated node embedding of the node to generate a respective de-noising prediction for the node that characterizes a de-noised feature representation for the node that does not include the noise used to generate the modified feature representation of the node; and
determining an update to current values of neural network parameters of the neural network to optimize an objective function that measures errors in the de-noising predictions for the nodes.

22. A system comprising:
one or more computers; and
one or more storage devices communicatively coupled to the one or more computers, wherein the one or more storage devices store instructions that, when executed by the one or more computers, cause the one or more computers to perform operations for training a neural network that includes one or more graph neural network layers, the operations comprising:
generating data defining a graph that comprises: (i) a set of nodes, (ii) a node embedding for each node, and (iii) a set of edges that each connect a respective pair of nodes, comprising:
obtaining a respective initial feature representation for each node;
generating a respective final feature representation for each node, wherein, for each of one or more of the nodes, the respective final feature representation is a modified feature representation that is generated from the respective feature representation for the node using respective noise; and
generating the data defining the graph using the respective final feature representations of the nodes;
processing the data defining the graph using one or more of the graph neural network layers of the neural network to generate a respective updated node embedding of each node;
processing, for each of one or more of the nodes having modified feature representations. the updated node embedding of the node to generate a respective de-noising prediction for the node that characterizes a de-noised feature representation for the node that does not include the noise used to generate the modified feature representation of the node; and
determining an update to current values of neural network parameters of the neural network to optimize an objective function that measures errors in the de-noising predictions for the nodes.
